(19) United States
(12) Patent Application Publication
(10) Pub. No.: US 2012/0245400 A1
(43) Pub. Date: Sep. 27, 2012

(54) PRODUCING ALPHA-OLEFINS

(76) Inventors: Thomas P. Clark, Midland, MI (US); Kevin A. Frazier, Midland, MI (US); Francis J. Timmers, Midland, MI (US); Brian W. Kolthammer, Lake Jackson, TX (US)

(21) Appl. No.: 13/514,148

(22) PCT Filed: Dec. 14, 2010
(86) PCT No.: PCT/US10/03160

§ 371 Date: Jun. 6, 2012

Related U.S. Application Data
(60) Provisional application No. 61/290,786, filed on Dec. 29, 2009.

(51) Int. Cl. C07C 4/06

(52) U.S. Cl. 585/324

(57) ABSTRACT

Methods for producing alpha-olefins. The methods include selectively isomerizing an alpha-olefin to a mixture of beta-olefins and ethenolyzing at least a portion of the mixture of beta-olefins to an alpha-olefin.

The present disclosure relates to methods for producing alpha-olefins (α-olefins), and in particular for selectively isomerizing an α-olefin to a mixture of beta-olefins (β-olefins) and ethenolyzing at least a portion of the β-olefin to an α-olefin.

Alpha-olefins with an even number of carbon atoms, e.g., 1-octene (C₈-H₁₆), 1-hexene (C₆H₁₂), etc., have a higher market value than α-olefins with an odd number of carbon atoms, e.g., 1-nonene (C₉H₁₈), 1-heptene (C₇H₁₄), etc. The even-numbered α-olefins have a higher market value, e.g., because they are the preferred industrial monomers for polymerization into polyolefins, and are available for purchase from many vendors. In contrast, odd-numbered α-olefins have limited industrial utility. However, odd-numbered α-olefins are used in a number of fields, e.g., hydrocarbon research. Hence, as described in the present disclosure, methods are useful for converting an odd-numbered α-olefin to an even-numbered α-olefin with one fewer carbon atom and for converting an even-numbered α-olefin to an odd-numbered α-olefin with one fewer carbon atom.

The present disclosure describes particular catalyst complexes that selectively isomerize a first α-olefin to β-olefin isomers of the first α-olefin. Following the α to β olefin isomerization reaction with ethenolysis, e.g., metathesis with excess ethylene (C₂H₄), a second α-olefin is produced, along with propylene (C₃H₆), by removing a terminal methyl group (—CH₃) from the β-olefin isomers to produce the second α-olefin. The second α-olefin has one fewer carbon atom than the first α-olefin. For instance, low market value 1-nonene is selectively isomerized to 2-nonene isomers and subsequent ethenolysis of the 2-nonene isomers produces higher valued 1-octene, along with marketable C₃H₆.

The present disclosure provides methods of utilizing a class of catalysts that isomerize α-olefins to produce olefins with a double carbon bond at an internal, rather than terminal, position. The class of catalysts has an unexpected ability to selectively induce isomerization at the 2-position to produce a mixture of cis and trans isomers of β-olefin, e.g., 2-nonene, 2-octene, 2-heptene, etc. Ethenolyzing the mixture of cis and trans isomers of the β-olefin produces a corresponding second α-olefin that has one fewer carbon atom, e.g., 1-octene, 1-heptene, 1-hexene, etc. Hence, the second α-olefin has one fewer methylene group (—CH₂) than the first α-olefin.

Advantages to utilizing the described methods include maintaining a preferred rate of the α to β olefin isomerization reaction at temperatures within a range of from 20 degrees Celsius (° C.) to 120° C. Using the described class of catalysts, raising the temperature of the isomerization reaction within the 20-120° C. temperature range increases the isomerization rate without significantly decreasing the selectivity of the isomerization of the double bond from the 1-position to the 2-position of the olefin. This stability in selectivity of the isomerization of the double bond from the 1-position to the 2-position of the olefin within the 20-120° C. temperature range contrasts with a decrease in isomerization selectivity occurring at temperatures above 120° C., as occurs with a variety of other catalysts. For instance, a variety of salts of Group VIII transition metals of the periodic table, e.g., a group of nine elements consisting of iron, ruthenium, osmium, cobalt, rhodium, iridium, nickel, palladium, and platinum, and/or sodium or potassium impregnated upon alumina or silica require temperatures above 120° C. and/or are non-selective for isomerizing an α-olefin at the 2-position.

Another advantage is that the preferred rate of the α to β olefin isomerization reaction is facilitated by utilizing catalyst complexes homogeneously, e.g., in solution, with the α-olefin being a substrate and the β-olefin being a product. Such homogeneous catalyst complexes better enable a preferred concentration of the catalyst, relative to the α-olefin substrate, to be readily achieved by adjustment of the concentration, e.g., in contrast to a fixed bed of heterogeneous catalyst. For instance, a first α-olefin is exposed to the homogeneous catalyst complex, e.g., as detailed below, utilizing the homogeneous catalyst complex in a mole percentage (mol %) within a range of from 0.001% to 10.0% relative to moles of the first α-olefin. As an alternative, the α to β olefin isomerization can be performed heterogeneously, e.g., with the α-olefin being exposed to a solid phase catalyst complex on the fixed bed.

In addition to selectivity of the α to β olefin isomerization reaction, conversion of the α-olefin with the homogeneous complexes to the corresponding β-olefin is at least 90%, as measured on a mol % basis, under certain conditions. This selectivity and conversion reduces a requirement for separation of an unreacted portion of the first α-olefin and undesired isomers prior to exposure to a concentration of ethylene sufficient to induce the ethenolysis reaction, as compared to not exposing the first α-olefin to a described homogeneous catalyst complex.

The homogeneous catalyst complex is at least one of: organometallic halides having two cyclopentadienyl rings, including substitution of the halide moiety with pseudo-halide groups, alkoxides, mesylates, triflates, dihydrocarbylamide, alkyls, and hydrides; metallocenes having two cyclopentadienyl rings; derivatives of organometallic halides and metallocenes having the cyclopentadienyl rings independently substituted with a number of hydrocarbyl groups; and ansa metallocenes, which are derivatives of metallocenes having an intramolecular bridge between the two cyclopentadienyl rings. Having the hydrocarbyl groups includes having a methyl and/or a phenyl group, and where adjacent hydrocarbyl groups form a cyclic ring, including an indenide and/or a tetrahydroindenide group. The derivatives of metallocenes include having an ethylene bis(indenyl) metal halide and a dimethylsilyl bis(indenyl) metal halide.

Metallocenes are a subset of a broader class of organometallic compounds that are also known as sandwich compounds. A metallocene has a general formula of (C₅H₅)₂M consisting of two cyclopentadienyl anions, e.g., Cp, which corresponds to one (C₅H₅) ring, bound to a metal atom (M) between the two rings. The Cp rings are aromatically stabilized with non-localized double bonding between the five carbon atoms.

The homogeneous catalyst complex includes a metal moiety based on at least one of iron (Fe), niobium (Nb), and titanium (Ti). Hence, the methods include utilizing 2,6-bis[1-(2,6-di-isopropylphenylimino)ethyl] pyridineiron (II) dichloride (prepared at The Dow Chemical Company (TDCC)), bis-cyclopentadienyl niobium (IV) dichloride (prepared at TDCC), and bis-cyclopentadienyl titanium dichloride (produced by Strem Chemicals, Inc.).

The methods include selectively isomerizing the first α-olefin to a β-olefin with the first α-olefin either being in admixture with an inert solvent, e.g., benzene, toluene, Isopar, hexanes, etc., or the first α-olefin serving both as solvent and reactant.

The methods include a precursor step of activating the homogeneous catalyst complex with at least one compound selected from a group that includes isobutyl aluminums and other aluminum alkyls, aluminum hydrides, organozinc compounds, organomagnesium compounds, trialkyl boranes, and borohydrides. Exposure to a sufficient concentration of at least one of these compounds increases the isomerization rate of the homogeneous catalyst complex relative to a rate obtained in the absence of such activation. For example, triisobutyl aluminum (TIBA), e.g., 1-10 molar equivalents relative to the homogeneous catalyst complex, is added to react with the homogeneous catalyst complex in order to activate the catalyst to isomerize the α-olefin at the preferred rate. As an alternative to forming an activated catalyst complex in situ by mixing the TIBA with the homogeneous catalyst complex, an admixture of the TIBA and the homogeneous catalyst complex can be prepared and isolated prior to introduction to the isomerization reaction. Some homogeneous catalyst complexes, e.g., those that include hydrides as part of the structure, already demonstrate sufficient isomerization activity without exposure to any of these compounds.

### Example (Ex) 1

A nuclear magnetic resonance (NMR) experiment using a 300 megahertz NMR instrument is run with an α-olefin, in this case 1-octene. 100 milliliters (ml) of 1-octene (20 milligrams (mg)) and 50 ml of TIBA (10 mg) are mixed in a 1.5 ml NMR tube with approximately 1.0 ml of benzene (C₆H₆), which has hydrogen atoms replaced by deuterium atoms (C₆D₆), used as solvent. NMR analysis confirms that there is no interaction/reaction of these components, such that the solvent is demonstrated to be inert. A catalytic amount of bis-cyclopentadienyl Nb (IV) dichloride (1 mg, 3.4 micromoles (μmol)), is added to the NMR tube and is shaken to create a homogeneous solution. The NMR tube is left at ambient temperature, e.g., 20-25° C., overnight in a glovebox, subsequent to which NMR analysis is performed on reaction products.

Analysis of an NMR spectrum shows that peaks corresponding to the 1-octene terminal olefin are largely replaced with peaks indicative of an internal olefin. That is, a 1.59 part per million (ppm) doublet of peaks, along with a smaller 1.57 ppm doublet of peaks, are consistent with both cis and trans isomers of 2-octene. There appears to be no significant binding of octyl groups, e.g., molecules having eight carbon atoms, to the aluminum (Al) because the 0.27 ppm peak corresponding to TIBA is unchanged from TIBA analyzed prior to the reaction.

The reaction is quenched with methanol (CH₄O) and the admixture of reagents and products is analyzed by gas chromatography (GC). Perform GC using an Alltech Econo-Cap™ EC-1 30 meter column with a flow rate of 1.0 ml/min and held at 40-4520 C. for 20 min, then ramped to 250° C. at 20° C./min and an EZ Chrom™ program. The NMR spectrum and GC histogram data are consistent with the products of the Nb complex catalyzed experiment being conversion of the original 1-octene to the cis and trans isomers of 2-octene.

### Ex 2

The 2,6-bis[1-(2,6-di-isopropylphenylimino)ethyl] pyridine Fe (II) dichloride catalyst complex (8.3 mg, 13.7 μmol) is placed in a 20 ml glass vial with a Teflon-coated stirbar. Toluene (C₆H₅CH₃) (1.5 ml) is added to the vial, followed by 1-octene (0.25 ml, 1.59 millimoles (mmol)). TIBA (30 mg, 0.16 mmol) is then added to the vial. The vial is capped and stirred for 2.75 hours (hrs) at 70° C. in a glovebox. The reaction is cooled and heptane (C₇H₁₆) (0.25 ml, 1.71 mmol) is added as an internal standard. CH₄O is added slowly to quench the reaction. An aliquot is removed from the vial and filtered through a plug of silica (SiO₂) gel with methylene chloride (CH₂Cl₂). The product mixture is analyzed by GC, which shows yields in mol % of: 2-octene=65%, 1-octene=11%, octane (C₆H₁₈)=8.9%, and 3- and 4-octenes=4.1%.

### Ex 3

Commercial 1-nonene (purchased from Trust Chemical Industries (TCI)), as analyzed by GC, shows the following mol % of nonene isomers: nonane (C₉H₂₀ )=2.1%, 1-nonene=97%, 2-nonene=0.24%, 3-nonene=0.23%, and 4-nonene=0.39%. 20 ml of the nonene is exposed to a sodium/potassium alloy and filtered through 11% triethylaluminum ((C₂H₅)₃Al) on SiO₂ to remove potential water (H₂O) and trace polar impurities. A resulting purified nonene is filtered through a 0.45 micron polytetrafluoroethylene (PTFE) syringe frit to remove residual silica particles. The bis-cyclopentadienyl titanium dichloride catalyst complex (2.2 mg, 8.8 μmol) is placed in a 20 ml glass reaction vial. A PTFE-coated stirbar is added. The solid catalyst complex is mixed with dodecane (CH₃(CH₂)₁₀CH₃) (0.25 ml, 1.10 mmol) as an internal standard. In a separate vial, TIBA (18 mg, 0.10 mmol) is dissolved in 1-nonene (2.2 ml, 12.7 mmol). The TIBA and 1-nonene solution is added to the reaction vial and the resulting admixture is stirred until homogeneity. The admixture is sealed with a PTFE-lined cap and stirred in an Al heating block at 90° C. in a nitrogen (N₂) purged glovebox.

Aliquots of the reaction are removed at 15, 45, 60, and 80 minutes (min). The aliquots are diluted with CH₂Cl₂ and quenched with CH₄O. The product mixture in the 45 min aliquot is analyzed by GC, which shows yields in mol % of: nonane=2.1%, 1-nonene=3.6%, 2-nonene=92%, 3-nonene=0.54%, and 4-nonene=0.48%.

### Ex 4

The isomerization reaction is run in a N₂ purged glovebox. H₂O and oxygen (O₂) are removed from 1-octene by passing the liquid through activated alumina (e.g., Al₂O₃) and copper oxide (CuO) on the alumina. Dicyclopentadienyltitanium dichloride (4.0 mg, 16 μmol) catalyst complex is placed in a 20 ml glass reaction vial and is suspended in 1-octene. TIBA (50 mg, 0.28 mmol) is added to the reaction vial. A PTFE-coated stirbar is added and the reaction vial is sealed with a PTFE-lined cap. The reaction is placed in an Al heating block at 75° C. and stirred overnight to put the catalyst into solution. After 15 hrs, the solution is cooled. An aliquot is transferred to GC vials, diluted with C₆H₅CH₃, and quenched with 0.1 ml of CH₄O. The aliquot is analyzed by GC. GC percentages as a mol % of the total eight carbon species are: octane=3.1%, 1-octene=2.8%, cis-2-octene=8.6%, trans-2-octene=83%, 3-octene=0.73%, 4-octene=0.06%. Hence, the cumulative mol % of the mixture of cis and trans isomers of 2-octene is 91.6%.

After purification to remove metals, e.g., the catalyst complex and TIBA, a low O₂, e.g., less than 1 ppm O₂, glove box is used to load reagents and perform the reaction. An 80 ml glass pressure vessel, plumbed to a C₂H₄ line, is loaded with 0.575 g of the mixture of 2-octene isomers and 20 ml of C₆H₅CH₃. A magnetic stir bar stirs the admixture and the admixture is heated to 85° C. with a heat block. The mixture is stirred at 85° C. for 15 min. 0.010 g of propyl acetate (C₅H₁₀O₂) in C₆H₅CH₃ is then loaded to the reaction vessel. Six mg of a tungsten oxychloride (WOCl₄)-diethyl ether (CH₃CH₂—O—CH₂CH₃) catalyst in C₆H₅CH₃ and 13 mg of 25% ethylaluminum dichloride (C₂H₅AlCl₂) in C₆H₅CH₃ are also loaded to the reactor vessel. The reactor vessel is flushed three times with C₂H₄ before pressurizing to 380 kiloPascals (kPa) with C₂H₄. The mixture is stirred under C₂H₄ pressure for 2 hrs.

After the 2 hr reaction time, the pressure on the reactor vessel is 400 kPa. The reactor is then vented and the reaction is quenched with 5 ml of 2-propanol ((CH₃)₂CHOH). The resulting solution is diluted and analyzed by GC. Analysis indicates the formation of 1-heptene with a conversion of 27% under these conditions. No other heptene isomers are detected in the mixture. Catalysts other than the WOCl₄—CH₃CH₂—O—CH₂CH₃ catalyst have a potential for yielding a conversion higher than 27%. These catalysts include a mixture of tungsten hexachloride (WCl₆), C₂H₅AlCl₂, and ethanol (C₂H₅OH) as a homogeneous catalyst and metal oxides such as tungsten oxide (WO₃), cobalt oxide-molybdenum oxide (CoO—MoO₃), and/or rhenium oxide (Re₂O₇) on supports of AlO₃ or SiO₂ as heterogeneous catalysts, among other potential catalysts.

1. A method for producing alpha-olefins comprising:
converting a first alpha-olefin of either a 1-octene or a 1-nonene, each without polar impurities, to isomers of beta-olefins of either 2-octene or 2-nonene, respectively, with a homogeneous catalyst complex to achieve a greater than 90% conversion of the first alpha-olefin to the isomers of beta-olefins, and
ethenolyzing at least a portion of the isomers of beta-olefins to propylene and
a second alpha-olefin having one less methylene group than the first alpha-olefin.

2. The method of claim 1, wherein the homogeneous catalyst complex is a metallocene for catalytically converting the first alpha-olefin to isomers of beta-olefins.

3-5. (canceled)

6. The method of claim 1, wherein the homogeneous catalyst complex includes a metal moiety based on titanium.

7. The method of claim 1, further comprising a precursor step of activating the homogeneous catalyst complex with at least one compound selected from a group that includes:
isobutyl aluminums and other aluminum alkyls;
aluminum hydrides;
organozinc compounds;
organomagnesium compounds;
trialkyl boranes; and
borohydrides.

8. The method of claim 1, wherein the alpha-olefin is in an admixture with an inert solvent.

9. The method of claim 1, wherein converting the first alpha-olefin includes converting the first alpha-olefin without polar impurities to isomers of beta-olefins with the homogeneous catalyst complex at a temperature within a range of from 20° C. to 120° C.

10. The method of claim 1, wherein converting the first alpha-olefin includes removing polar impurities from the first alpha-olefin.
